(19) United States
(12) Patent Application Publication
(10) Pub. No.: US 2011/0092704 A1
(43) Pub. Date: Apr. 21, 2011

(54) Preparation of triazospiro compounds

(76) Inventors: Parvis Gharagozloo, Pennington, NJ (US); Gary Lee, West Windsor, NJ (US); R. Richard Goehring, Pipersville, PA (US)

(21) Appl. No.: 10/687,245

(22) Filed: Oct. 16, 2003

Related U.S. Application Data
(60) Provisional application No. 60/419,305, filed on Oct. 17, 2002.
(60) Provisional application No. 60/419,600, filed on Oct. 18, 2002.

(51) Int. Cl. C07D 471/10

(52) U.S. Cl. 546/20

(57) ABSTRACT

The present invention relates to processes for producing triazospiro compounds having the formula (IV):

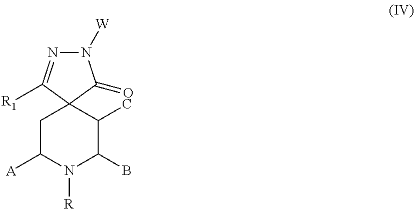

wherein A, B, C, R, R₁, and W are as defined herein.

### FIELD OF THE INVENTION

The present invention relates to processes for producing triazospiro compounds.

### BACKGROUND OF THE INVENTION

A number of therapeutically useful spiro compounds and process of making the same are described in the art.

For example, U.S. Pat. No. 5,852,029, to Fisher et al., describes the use and preparation of certain aza spiro compounds which act on the cholinergic system. U.S. Pat. No. 5,633,247, to Baldwin et al., describes certain nitrogen-containing spirocycles which act as antiarrhythmic agents.

U.S. Pat. No. 6,277,991, to Hohlweg et al., describes the use and preparation of certain triaza-spiro compounds for the treatment of migraine, non-insulin dependent diabetes mellitus (type II diabetes), sepsis, inflammation, incontinence or vasomotor disturbances, in particular the peripheral vasomotor effects known as hot flushes or hot flashes.

U.S. Pat. No. 4,220,773 to Wiezer et al., describes a process for the manufacture of certain aza-spiro decanes.

U.S. patent application Ser. No. 10/126,506, filed Apr. 18, 2002, discloses certain spiropyrazole compounds which exhibit affinity for the ORL1 receptor and further discloses certain spiropyrazole compounds which exhibit affinity for the ORL1 receptor and one or more of the μ, δ or κ receptors. Certain compounds described in U.S. patent application Ser. No. 10/126,506 are useful for treating a patient suffering from chronic or acute pain. The application also describes certain spiropyrazole compounds disclosed therein as being useful as analgesics, anti-inflammatories, diuretics, anesthetics and neuroprotective agents, anti-hypertensives, anti-anxioltics, agents for appetite control, hearing regulators, anti-tussives, anti-asthmatics, modulators of locomotor activity, modulators of learning and memory, regulators of neurotransmitter and hormone release, kidney function modulators, anti-depressants, agents to treat memory loss due to Alzheimer's disease or other dementias, anti-epileptics, anti-convulsants, agents to treat withdrawal from alcohol and drugs of addiction, agents to control water balance, agents to control sodium excretion and agents to control arterial blood pressure disorders and methods for administering said compounds.

There exists a need in the art for improved processes for producing triazospiro compounds.

### OBJECTS AND SUMMARY OF THE INVENTION

It is accordingly an object of certain embodiments of the present invention to provide a process for the preparation of triazospiro compounds.

It is an object of certain embodiments of the present invention to provide a process for the preparation of triazospiro compounds which exhibit affinity for the ORL1 receptor.

It is an object of certain embodiments of the present invention to provide a process for the preparation of triazospiro compounds which exhibit affinity for the ORL1 receptor and one or more of the μ, δ or κ receptors.

It is an object of certain embodiments of the present invention to provide a process for the preparation of triazospiro compounds for treating a patient suffering from chronic or acute pain.

It is an object of certain embodiments of the present invention to provide a process for the preparation of triazospiro compounds useful as analgesics, anti-inflammatories, diuretics, anesthetics and neuroprotective agents, anti-hypertensives, anti-anxioltics, agents for appetite control, hearing regulators, anti-tussives, anti-asthmatics, modulators of locomotor activity, modulators of learning and memory, regulators of neurotransmitter and hormone release, kidney function modulators, anti-depressants, agents to treat memory loss due to Alzheimer's disease or other dementias, anti-epileptics, anti-convulsants, agents to treat withdrawal from alcohol and drugs of addiction, agents to control water balance, agents to control sodium excretion or agents to control arterial blood pressure disorders.

Other objects and advantages of the present invention will become apparent from the following detailed description thereof.

The present invention is directed in part to a process for preparing compounds having the general formula (IV):

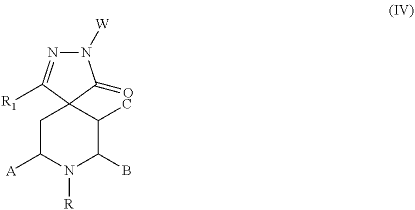

wherein

W is hydrogen, C₁₋₁₀ alkyl, C₃₋₁₂ cycloalkyl, C₃₋₁₂ cycloalkylC₁₋₄alkyl-, C₁₋₁₀ alkoxy, C₃₋₁₂ cycloalkoxy-, C₁₋₁₀ alkyl substituted with 1-3 halogen, C₃₋₁₂ cycloalkyl substituted with 1-3 halogen, C₃₋₁₂ cycloalkylC₁₋₄ alkyl- substituted with 1-3 halogen, C₁₋₁₀ alkoxy substituted with 1-3 halogen, C₃₋₁₂ cycloalkoxy- substituted with 1-3 halogen, —COOV₁, —C₁₋₄COOV₁, —CH₂OH, —SO₂N(V₁)₂, hydroxyC₁₋₁₀alkyl-, hydroxyC₃₋₁₀cycloalkyl-, cyanoC₁₋₁₀alkyl-, cyanoC₃₋₁₀cycloalkyl-, —CON(V₁)₂, NH₂SO₂C₁₋₄ alkyl-, NH₂SOC₁₋₄alkyl-, sulfonylaminoC₁₋₁₀alkyl-, diaminoalkyl-, -sulfonylC₁₋₄alkyl, a 6-membered heterocyclic ring, a 6-membered heteroaromatic ring, a 6-membered heterocyclicC₁₋₄alkyl-, a 6-membered heteroaromaticC₁₋₄ alkyl-, a 6-membered aromatic ring, a 6-membered aromaticC₁₋₄ alkyl-, a 5-membered heterocyclic ring optionally substituted with an oxo or thio, a 5-membered heteroaromatic ring, a 5-membered heterocyclicC₁₋₄alkyl- optionally substituted with an oxo or thio, a 5-membered heteroaromaticC₁₋₄alkyl-, —C₁₋₅(═O)W₁, —C₁₋₅(═NH)W₁, —C₁₋₅NHC(═O)W₁, —C₁₋₅NHS(═O)₂W₁, —C₁₋₅NHS(═O)W₁, wherein W₁ is hydrogen, C₁₋₁₀ alkyl, C₃₋₁₂ cycloalkyl, C₁₋₁₀ alkoxy, C₃₋₁₂ cycloalkoxy, —CH₂OH, amino, C₁₋₄alkylamino-, diC₁₋₄alkylamino-, or a 5-membered heteroaromatic ring optionally substituted with 1-3 lower alkyl;

wherein each V₁ is independently selected from H, C₁₋₆ alkyl, C₃₋₆ cycloalkyl, benzyl and phenyl;

A, B and C are independently hydrogen, C₁₋₁₀ alkyl, C₃₋₁₂ cycloalkyl, C₁₋₁₀ alkoxy, C₃₋₁₂ cycloalkoxy, —CH₂OH, —NHSO₂, hydroxyC₁₋₁₀alkyl-, aminocarbonyl-, C₁₋₄alkylaminocarbonyl-, diC₁₋₄alkylaminocarbonyl-, acylamino-, acylaminoalkyl-, amide, sulfonylaminoC₁₋₁₀alkyl-, or A-B can together form a C₂₋₆ bridge, or B-C can together form a C₃₋₇ bridge, or A-C can together form a C₁₋₅ bridge;

R is —Z—R₂; wherein Z is selected from the group consisting of a bond, straight or branched C₁₋₆ alkylene, —NH—, —CH₂O—, —CH₂NH—, —CH₂N(CH₃)—, —NHCH₂—, —CH₂CONH—, —NHCH₂CO—, —CH₂CO—, —COCH₂—, —CH₂COCH₂—, —CH(CH₃)—, —CH═, —O— and —HC═CH—, wherein the carbon and/or nitrogen atoms are unsubstituted or substituted with one or more lower alkyl, hydroxy, halo or alkoxy group;

R₂ is selected from the group consisting of hydrogen, C₁₋₁₀ alkyl, C₃₋₁₂cycloalkyl, C₂₋₁₀alkenyl, amino, C₁₋₁₀alkylamino-, C₃₋₁₂cycloalkylamino-, —COOV₁, —C₁₋₄COOV₁ cyano, cyanoC₁₋₁₀alkyl-, cyanoC₃₋₁₀cycloalkyl-, NH₂SO₂—, NH₂SO₂C₁₋₄alkyl-, NH₂SOC₁₋₄alkyl-, aminocarbonyl-, C₁₋₄alkylaminocarbonyl-, diC₁₋₄alkylaminocarbonyl-, benzyl, C₃₋₁₂ cycloalkenyl-, a monocyclic, bicyclic or tricyclic aryl or heteroaryl ring, a hetero-monocyclic ring, a hetero-bicyclic ring system, and a spiro ring system of the formula (V):

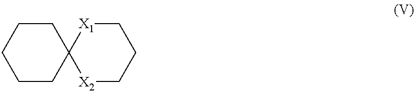

wherein X₁ and X₂ are independently selected from the group consisting of NH, O, S and CH₂; and wherein said alkyl, cycloalkyl, alkenyl, C₁₋₁₀alkylamino—, C₃₋₁₂cycloalkylamino-, or benzyl of R₁ is optionally substituted with 1-3 substituents selected from the group consisting of halogen, hydroxy, C₁₋₁₀ alkyl, C₁₋₁₀ alkoxy, nitro, trifluoromethyl-, cyano, —COOV₁, —C₁₋₄COOV₁, cyanoC₁₋₁₀alkyl-, —C₁₋₅NHS(═O)₂W₁, —C₁₋₅NHS(═O)W₁, a 5-membered heteroaromaticC₀₋₄alkyl-, phenyl, benzyl, benzyloxy, said phenyl, benzyl, and benzyloxy optionally being substituted with 1-3 substituents selected from the group consisting of halogen, C₁₋₁₀ alkyl-, C₁₋₁₀ alkoxy-, and cyano; and wherein said C₃₋₁₂ cycloalkyl, C₃₋₁₂ cycloalkenyl, monocyclic, bicyclic or tricyclic aryl, heteroaryl ring, hetero-monocyclic ring, hetero-bicyclic ring system, or spiro ring system of the formula (V) is optionally substituted with 1-3 substituents selected from the group consisting of halogen, C₁₋₁₀ alkyl, C₁₋₁₀ alkoxy, nitro, trifluoromethyl-, phenyl, benzyl, phenyloxy and benzyloxy, wherein said phenyl, benzyl, phenyloxy or benzyloxy is optionally substituted with 1-3 substituents selected from the group consisting of halogen, C₁₋₁₀ alkyl, C₁₋₁₀ alkoxy, and cyano;

R₁ is selected from the group consisting of C₁₋₈ alkyl, 5-8 membered cycloalkyl, 5-8 membered heterocyclic or a 6 membered aromatic or heteroaromatic group; and R₁ being substituted with (D)ₙ, wherein n is an integer from 0 to 3, and wherein D is selected from the group consisting of hydrogen, C₁₋₁₀ alkyl, C₃₋₁₂ cycloalkyl and halogen, said alkyl or cycloalkyl optionally substituted with an oxo, amino, alkylamino or dialkylamino group;

and pharmaceutically acceptable salts thereof and solvates thereof.

In certain preferred embodiments, R₁ is phenyl or a 6 membered heteroaromatic group containing 1-3 nitrogen atoms.

In certain preferred embodiments, the R₂ alkyl is methyl, ethyl, propyl, butyl, pentyl, or hexyl.

In certain preferred embodiments, the R₂ cycloalkyl is cyclohexyl, cycloheptyl, cyclooctyl, cyclononyl, cyclodecyl, or norbornyl.

In other preferred embodiments, the R₂ bicyclic ring system is naphthyl. In other preferred embodiments, the R₂ bicyclic ring system is tetrahydronaphthyl, or decahydronaphthyl and the R₂ tricyclic ring system is dibenzocycloheptyl. In other preferred embodiments R₂ is phenyl or benzyl.

In other preferred embodiments, the R₂ bicyclic aromatic ring is a 10-membered ring, preferably quinoline or naphthyl.

In other preferred embodiments, the R₂ bicyclic aromatic ring is a 9-membered ring, preferably indenyl.

In certain embodiments, Z is a bond, methyl, or ethyl.

In certain embodiments, the Z group is maximally substituted as not to have any hydrogen substitution on the base Z group. For example, if the base Z group is —CH₂—, substitution with two methyl groups would remove hydrogens from the —CH₂— base Z group.

In other preferred embodiments, n is 0.

In certain embodiments, X₁ and X₂ are both O.

In certain embodiments, W is —CH₂C═ONH₂, —C(NH)NH₂, pyridylmethyl, cyclopentyl, cyclohexyl, furanylmethyl, —C═OCH₃, —CH₂CH₂NHC═OCH₃, —SO₂CH₃, CH₂CH₂NHSO₂CH₃, furanylcarbonyl-, methylpyrrolylcarbonyl-, diazolecarbonyl-, azolemethyl-, trifluoroethyl-, hydroxyethyl-, cyanomethyl-, oxo-oxazolemethyl-, or diazolemethyl-.

In certain embodiments, R is cyclohexylethyl-, cyclohexylmethyl-, cyclopentylmethyl-, dimethylcyclohexylmethyl-, phenylethyl-, pyrrolyltrifluoroethyl-, thienyltrifluoroethyl-, pyridylethyl-, cyclopentyl-, cyclohexyl-, methoxycyclohexyl-, tetrahydropyranyl-, propylpiperidinyl-, indolylmethyl-, pyrazoylpentyl-, thiazolylethyl-, phenyltrifluoroethyl-, hydroxyhexyl-, methoxyhexyl-, isopropoxybutyl-, hexyl-, or oxocanylpropyl-.

In certain embodiments, at least one of R or W is —CH₂COOV₁, tetrazolylmethyl-, cyanomethyl-, NH₂SO₂-methyl-, NH₂SOmethyl-, aminocarbonylmethyl-, C₁₋₄alkylaminocarbonylmethyl-, or diC₁₋₄ alkylaminocarbonylmethyl-.

In certain embodiments, R is 3,3 diphenylpropyl optionally substituted at the 3 carbon of the propyl with —COOV₁, tetrazolylC₀₋₄alkyl-, cyano-, aminocarbonyl-, C₁₋₄alkylaminocarbonyl-, or diC₁₋₄ alkylaminocarbonyl-.

In certain embodiments, A is hydrogen. In certain embodiments, B is hydrogen. In certain embodiments, C is hydrogen. In certain embodiments, A and B are hydrogen. In certain embodiments, A and C are hydrogen. In certain embodiments, B and C are hydrogen. In certain preferred embodiments, A, B and C are hydrogen.

In certain embodiments, A and B are hydrogen and C is selected from the group consisting of C₁₋₄ alkyl and hydroxyC₁₋₄alkyl. In certain embodiments, A and C are hydrogen and B is selected from the group consisting of C₁₋₄ alkyl and hydroxyC₁₋₄alkyl. In certain embodiments, B and C are hydrogen and A is selected from the group consisting of C₁₋₄ alkyl and hydroxyC₁₋₄alkyl.

In alternate embodiments, R can be

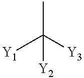

wherein

Y₁ is R₃—(C₁-C₁₂)alkyl, R₄-aryl, R₅-heteroaryl, R₆—(C₃-C₁₂)cyclo-alkyl, R₇—(C₃-C₇)heterocycloalkyl, —CO₂(C₁-C₆)alkyl, CN or —C(O)NR₈R₉; Y₂ is hydrogen or Y₁; Y₃ is hydrogen or (C₁-C₆)alkyl; or Y₁, Y₂ and Y₃, together with the carbon to which they are attached, form one of the following structures:

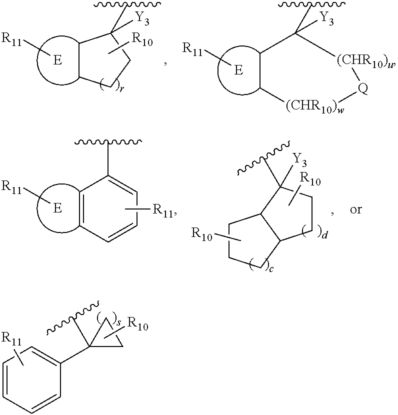

wherein r is 0 to 3; w and u are each 0-3, provided that the sum of w and u is 1-3; c and d are independently 1 or 2; s is 1 to 5; and ring E is a fused R₄-phenyl or R₅-heteroaryl ring;

R₁₀ is 1 to 3 substituents independently selected from the group consisting of H, (C₁-C₆)alkyl, —OR₈, —(C₁-C₆)alkyl-OR₈, —NR₈R₉ and —(C₁-C₆)alkyl-NR₈R₉;

R₁₁ is 1 to 3 substituents independently selected from the group consisting of R₁₀; —CF₃, —OCF₃, NO₂ and halo, or R₁₁ substituents on adjacent ring carbon atoms may together form a methylenedioxy or ethylenedioxy ring;

R₈ and R₉ are independently selected from the group consisting of hydrogen, (C₁-C₆) alkyl, (C₃-C₁₂)cycloalkyl, aryl and aryl(C₁-C₆)alkyl;

R₃ is 1 to 3 substituents independently selected from the group consisting of H, R₄-aryl, R₆—(C₃-C₁₂)cycloalkyl, R₅-heteroaryl, R₇—(C₃-C₇)heterocycloalkyl, —NR₈R₉, —OR₁₂ and —S(O)₀₋₂R₁₂;

R₆ is 1 to 3 substituents independently selected from the group consisting of H, (C₁-C₆)alkyl, R₄-aryl, —NR₈R₉, —OR₁₂ and —SR₁₂;

R₄ is 1 to 3 substituents independently selected from the group consisting of hydrogen, halo, (C₁-C₆)alkyl, R₁₃-aryl, (C₃-C₁₂)cycloalkyl, —CN, —CF₃, —OR₈, —(C₁-C₆)alkyl-OR₈, —OCF₃, —NR₈R₉, —(C₁-C₆)alkyl —NR₈R₉, —NHSO₂R₈, —SO₂N(R₁₄)₂, —SO₂R₈, —SOR₈, —SR₈, —NO₂, —CONR₈R₉, —NR₉COR₈, —COR₈, —COCF₃, —OCOR₈, —OCO₂R₈; —COOR₈, —(C₁-C₆)alkyl-NHCOOC(CH₃)₃, —(C₁-C₆)alkyl-NHCOCF₃, —(C₁-C₆)alkyl-NHSO₂—(C₁-C₆)alkyl, —(C₁-C₆)alkyl-NHCONH—(C₁-C₆)-alkyl and

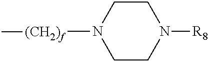

wherein f is 0 to 6; or R₄ substituents on adjacent ring carbon atoms may together form a methylenedioxy or ethylenedioxy ring;

R₅ is 1 to 3 substituents independently selected from the group consisting of hydrogen, halo, (C₁-C₆)alkyl, R₁₃-aryl, (C₃-C₁₂)cycloalkyl, —CN, —CF₃, —OR₈, —(C₁-C₆)alkyl-OR₈, —OCF₃, —NR₈R₉, —(C₁-C₆)alkyl-NR₈R₉, —NHSO₂R₈, —SO₂N(R₁₄)₂, —NO₂, —CONR₈R₉, —NR₉COR₈, —COR₈, —OCOR₈, —OCO₂R₈ and —COOR₈;

R₇ is H, (C₁-C₆)alkyl, —OR₈, —(C₁-C₆)alkyl-OR₈, —NR₈R₉ or —(C₁-C₆)alkyl-NR₈R₉;

R₁₂ is H, (C₁-C₆)alkyl, R₄-aryl, —(C₁-C₆)alkyl-OR₈, —(C₁-C₆)alkyl-NR₈R₉, —(C₁-C₆)alkyl-SR₈, or aryl(C₁-C₆)alkyl;

R₁₃ is 1-3 substituents independently selected from the group consisting of H, (C₁-C₆)alkyl, (C₁-C₆)alkoxy and halo;

R₁₄ is independently selected from the group consisting of H, (C₁-C₆)alkyl and R₁₃—C₆H₄—CH₂—.

In certain embodiments of the present invention the triazospiro compounds produced by the processes of the present invention exhibit affinity for the ORL1 receptor.

It certain embodiments of the present invention the triazospiro compounds produced by the processes of the present invention exhibit affinity for the ORL1 receptor and one or more of the μ, δ or κ receptors.

In certain embodiments of the present invention the triazospiro compounds produced by the processes of the present invention are useful for treating a patient suffering from chronic or acute pain.

In certain embodiments of the present invention the triazospiro compounds produced by the processes of the present invention are useful as analgesics, anti-inflammatories, diuretics, anesthetics and neuroprotective agents, anti-hypertensives, anti-anxioltics, agents for appetite control, hearing regulators, anti-tussives, anti-asthmatics, modulators of locomotor activity, modulators of learning and memory, regulators of neurotransmitter and hormone release, kidney function modulators, anti-depressants, agents to treat memory loss due to Alzheimer's disease or other dementias; anti-epileptics, anti-convulsants, agents to treat withdrawal from alcohol and drugs of addiction, agents to control water balance, agents to control sodium excretion and agents to control arterial blood pressure disorders.

In certain embodiments, the invention is directed to a compound of formula (IV) wherein R₁ is hydrogen and A, B, C, R, and W are as disclosed above; a pharmaceutical composition comprising a compound of formula (IV) wherein R₁ is hydrogen and A, B, C, R, and W are as disclosed above and at least one pharmaceutically acceptable excipient; and methods of treating a patient comprising administering to a patient a compound of formula (IV) wherein R₁ is hydrogen and A, B, C, R, and W are as disclosed above which exhibits affinity to the ORL1 receptor.

As used herein, the term “alkyl” means a linear or branched saturated aliphatic hydrocarbon group having a single radical and 1-10 carbon atoms. Examples of alkyl groups include methyl, propyl, isopropyl, butyl, n-butyl, isobutyl, sec-butyl, tert-butyl, and pentyl. A branched alkyl means that one or more alkyl groups such as methyl, ethyl or propyl, replace one or both hydrogens in a —CH₂— group of a linear alkyl chain. The term “lower alkyl” means an alkyl of 1-3 carbon atoms.

The term “alkoxy” means an “alkyl” as defined above connected to an oxygen radical.

The term “cycloalkyl” means a non-aromatic mono- or multicyclic hydrocarbon ring system having a single radical and 3-12 carbon atoms. Exemplary monocyclic cycloalkyl rings include cyclopropyl, cyclopentyl, and cyclohexyl. Exemplary multicyclic cycloalkyl rings include adamantyl and norbornyl.

The term “alkenyl” means a linear or branched aliphatic-hydrocarbon group containing a carbon-carbon double bond having a single radical and 2-10 carbon atoms.

A “branched” alkenyl means that one or more alkyl groups such as methyl, ethyl or propyl replace one or both hydrogens in a —CH₂— or —CH═ linear alkenyl chain. Exemplary alkenyl groups include ethenyl, 1- and 2-propenyl, 1-, 2- and 3-butenyl, 3-methylbut-2-enyl, 2-propenyl, heptenyl, octenyl and decenyl.

The term “cycloalkenyl” means a non-aromatic monocyclic or multicyclic hydrocarbon ring system containing a carbon-carbon double bond having a single radical and 3 to 12 carbon atoms. Exemplary monocyclic cycloalkenyl rings include cyclopropenyl, cyclopentenyl, cyclohexenyl or cycloheptenyl. An exemplary multicyclic cycloalkenyl ring is norbornenyl.

The term “aryl” means a carbocyclic aromatic ring system containing one, two or three rings which may be attached together in a pendent manner or fused, and containing a single radical. Exemplary aryl groups include phenyl, naphthyl and acenaphthyl.

The term “heterocyclic” means cyclic compounds having one or more heteroatoms (atoms other than carbon) in the ring, and having a single radical. The ring may be saturated, partially saturated or unsaturated, and the heteroatoms may be selected from the group consisting of nitrogen, sulfur and oxygen. Examples of saturated heterocyclic radicals include saturated 3 to 6-membered hetero-monocyclic groups containing 1 to 4 nitrogen atoms, such as pyrrolidinyl, imidazolidinyl, piperidino, piperazinyl; saturated 3- to 6-membered hetero-monocyclic groups containing 1 to 2 oxygen atoms and 1 to 3 nitrogen atoms, such as morpholinyl; saturated 3- to 6-membered hetero-monocyclic groups containing 1 to 2 sulfur atoms and 1 to 3 nitrogen atoms, such as thiazolidinyl. Examples of partially saturated heterocyclic radicals include dihydrothiophene, dihydropyran, and dihydrofuran. Other heterocyclic groups can be 7 to 10 carbon rings substituted with heteroatoms such as oxocanyl and thiocanyl. When the heteroatom is sulfur, the sulfur can be a sulfur dioxide such as thiocanyldioxide.

The term “heteroaryl” means unsaturated heterocyclic radicals, wherein “heterocyclic” is as previously described. Exemplary heteroaryl groups include unsaturated 3 to 6 membered hetero-monocyclic groups containing 1 to 4 nitrogen atoms, such as pyrrolyl, pyridyl, pyrimidyl, and pyrazinyl; unsaturated condensed heterocyclic groups containing 1 to 5 nitrogen atoms, such as indolyl, quinolyl and isoquinolyl; unsaturated 3 to 6-membered hetero-monocyclic groups containing an oxygen atom, such as furyl; unsaturated 3 to 6 membered hetero-monocyclic groups containing a sulfur atom, such as thienyl; unsaturated 3 to 6 membered hetero-monocyclic groups containing 1 to 2 oxygen atoms and 1 to 3 nitrogen atoms, such as oxazolyl; unsaturated condensed heterocyclic groups containing 1 to 2 oxygen atoms and 1 to 3 nitrogen atoms, such as benzoxazolyl; unsaturated 3 to 6 membered hetero-monocyclic groups containing 1 to 2 sulfur atoms and 1 to 3 nitrogen atoms, such as thiazolyl; and unsaturated condensed heterocyclic group containing 1 to 2 sulfur atoms and 1 to 3 nitrogen atoms, such as benzothiazolyl. The term “heteroaryl” also includes unsaturated heterocyclic radicals, wherein “heterocyclic” is as previously described, in which the heterocyclic group is fused with an aryl group, in which aryl is as previously described. Exemplary fused radicals include benzofuran, benzdioxole and benzothiophene.

As used herein, the term “heterocyclicC₁₋₄alkyl”, “heteroaromaticC₁₋₄alkyl” and the like refer to the ring structure bonded to a C₁₋₄ alkyl radical.

All of the cyclic ring structures disclosed herein can be attached at any point where such connection is possible, as recognized by one skilled in the art.

As used herein, the term “patient” includes a human or an animal such as a companion animal or livestock.

As used herein, the term “halogen” includes fluoride, bromide, chloride, iodide or alabamide.

As used herein, the term “substituted hydrazine” is hydrazine with a substitution which, when used in reaction C as disclosed herein, results in a compound of formula IV with a W substituent as disclosed herein.

The W substituent can be substituted on the spiro ring of formula IV during reaction C as disclosed herein, e.g., by way of a substituted hydrazine, or can be included on the spiro ring by a separate reaction after formation of the spiro ring by reactions known to one skilled in the art. One skilled in the art would know which W substituents can be added by way of reaction with substituted hydrazine and which W substituents can be added by way of a separate reaction after formation of the spiro ring.

The compounds formed by the invention disclosed may be formed into a pharmaceutically acceptable salt of the compound. The pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, sulfate, phosphate and the like; organic acid salts such as formate, acetate, trifluoroacetate, maleate, fumarate, tartrate and the like; sulfonates such as methanesulfonate, benzenesulfonate, p-toluenesulfonate, and the like; amino acid salts such as arginate, asparaginate, glutamate and the like.

The compounds formed by the invention disclosed herein may be further formed into prodrugs. Prodrugs are considered to be any covalently bonded carriers which release the active parent drug in vivo.

The compounds formed by the invention disclosed herein are also meant to encompass the disclosed compounds being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Some of the compounds disclosed herein may contain one or more asymmetric centers and may thus give rise to enantiomers, diastereomers, and other stereoisomeric forms. The present invention is also meant to encompass all such possible forms as well as their racemic and resolved forms and mixtures thereof. When the compounds described herein contain olefinic double bonds or other centers of geometric asymmetry, and unless specified otherwise, it is intended to include both E and Z geometric isomers. All tautomers are intended to be encompassed by the present invention as well

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” refers to a carbon atom to which four different groups are attached.

The term “enantiomer” or “enantiomeric” refers to a molecule that is nonsuperimposable on its mirror image and hence optically active wherein the enantiomer rotates the plane of polarized light in one direction and its mirror image rotates the plane of polarized light in the opposite direction.

The term “racemic” refers to a mixture of equal parts of enantiomers and which is optically inactive.

The term “resolution” refers to the separation or concentration or depletion of one of the two enantiomeric forms of a molecule.

The term “modulate” as used herein with respect to the ORL-1 receptor means the mediation of a pharmacodynamic response (e.g., analgesia) in a subject from (i) inhibiting or activating the receptor, or (ii) directly or indirectly affecting the normal regulation of the receptor activity. Compounds which modulate the receptor activity include agonists, antagonists, mixed agonists/antagonists and compounds which directly or indirectly affect regulation of the receptor activity.

Certain preferred compounds prepared in accordance with the process of the invention include:
8-(4-propylcyclohexyl)-1-phenyl-2,3,8-triazospiro[4.5]decan-4-one;
8-(5-methylhex-2-yl)-1-phenyl-2,3,8-triazospiro[4.5]decan-4-one;
8-norbornyl-1-phenyl-2,3,8-triazospiro[4.5]decan-4-one;
8-(decahydro-2-naphthyl)-1-phenyl-2,3,8-triazospiro[4.5]decan-4-one;
8-(cyclooctylmethyl)-1-phenyl-2,3,8-triazospiro[4.5]decan-4-one;
8-(1,2,3,4-tetrahydro-2-naphthyl)-1-phenyl-2,3,8-triazospiro[4.5]decan-4-one;
8-[4-(2-propyl)-cyclohexyl]-1-phenyl-2,3,8-triazospiro[4.5]decan-4-one;
8-(1,3-dihydroinden-2-yl)-1-phenyl-2,3,8-triazospiro[4.5]decan-4-one;
8-[(naphth-2-yl-methyl)]-1-phenyl-2,3,8-triazospiro[4.5]decan-4-one;
8-(p-phenylbenzyl)-1-phenyl-2,3,8-triazospiro[4.5]decan-4-one;
8-[4,4-Bis(4-fluorophenyl)butyl]-1-phenyl-2,3,8-triazospiro[4.5]decan-4-one;
8-(benzyl)-1-phenyl-2,3,8-triazospiro[4.5]decan-4-one;
8-(10,11-Dihydro-5H-dibenzo[a,d]-cyclohepten-5-yl)-1-phenyl-2,3,8-triazospiro[4.5]decan-4-one;
8-(3,3-Bis(phenyl)propyl)-1-phenyl-2,3,8-triazospiro[4.5]decan-4-one;
8-(p-benzyloxybenzyl)-1-phenyl-2,3,8-triazospiro[4.5]decan-4-one;
8-(cyclooctylmethyl)-1-phenyl-2,3,8-triazospiro[4.]decan-4-one; and

pharmaceutically acceptable salts thereof and solvates thereof.

Another preferred compound is 8-(acenaphthen-9-yl)-1-phenyl-2,3,8-triazospiro[4.5]decan-4-one and pharmaceutically acceptable salts thereof and solvates thereof.

The present invention also provides use of any of the disclosed compounds in the preparation of a medicament for treating pain and other disease states modulated by an opioid receptor, e.g., the ORL-1 receptor.

### DETAILED DESCRIPTION

In accordance with certain embodiments of the present invention, the compound of the general formula (IV)

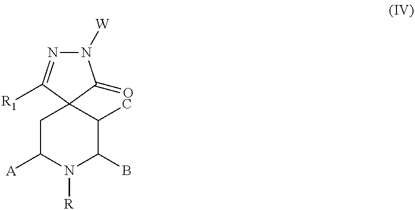

wherein R, R₁, A, B, C and W have the same meaning as mentioned above, is prepared generally with a compound of formula (III) to obtain a compound of formula (IV) through the following reaction scheme:

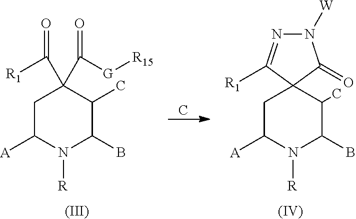

wherein R, R₁, A, B, C and W are as defined above, G is O or S and R₁₅ is selected from straight chained or branched C₁₋₁₀ alkyl, C₃₋₁₂ cycloalkyl, C₃₋₁₂cycloalkylC₁₋₁₀alkyl, aryl, heteroaryl, arylC₁₋₁₀alkyl or heteroarylC₁₋₁₀alkyl:

Reaction C is preferably a reduction and cyclization reaction. Preferably, in reaction C, the compound of formula (III) is reacted with hydrazine or substituted hydrazine (e.g., hydrazine hydrate) forming the compound of formula (IV).

In certain embodiments, it is preferable to employ 1 mole to excessive mole of the hydrazine or substituted hydrazine to 1 mole of a compound of the general formula (III).

In certain embodiments, the aforesaid reaction C proceeds in the absence of bases. In certain alternate embodiments, the aforesaid reaction C proceeds in the presence of a base. Certain bases which may be useful in accordance with this reaction include, for example and without limitation, alcohol solvents such as, methanol, ethanol, isopropyl alcohol, or n-butanol; ketone solvents, such as, cyclohexanone or methyl isobutyl ketone; hydrocarbon solvents, such as, benzene, toluene or xylene; halogenated hydrocarbons, such as, chlorobenzene or methylene chloride or dimethylformamide; and the like.

In certain embodiments, a catalyst may be used in reaction C. Suitable catalysts include for example, palladium catalysts, like palladium chloride, palladium acetate, palladium hydroxide, palladium oxide, palladium carbon, palladium hydroxide carbon, tetrakis(triphenylphosphine) palladium(0), dichlorobis(triphenylphosphine) palladium(II), or benzylchlorobis(triphenylphosphine) palladium(II); or nickel-phosphine catalysts. The amount of the catalyst is preferably 0.0001 to 0.5 parts by weight per 1 part by weight of formula III.

Reaction temperature is usually −20° C. to 150° C., preferably 0° C. to 100° C.

In certain embodiments of the present invention, the compound of the general formula (IV) as described above is prepared through the following reaction scheme:

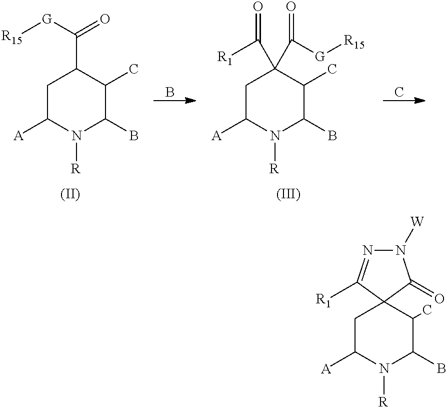

This process of the present invention includes subjecting formula (II) to a reaction B which is an acylation reaction.

In the acylation reaction, preferably the compound of formula (II):

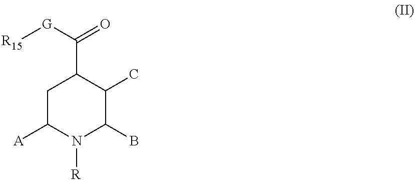

is subjected to an acylation reaction with an acid halide of the formula

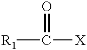

wherein R₁ is selected from the group as described above; and wherein X is a halogen, preferably Br or Cl; and preferably forming a compound of the formula (III) as described above wherein R, R₁, A, B, C, are selected from the groups as described above. Thereafter, formula (III) is subject to a reduction and cyclization reaction (reaction C). Preferably, in reaction C, the compound of formula (III) is reacted with hydrazine or substituted hydrazine (e.g., hydrazine hydrate), as described above, forming the compound of formula (IV).

In certain embodiments, R₁ is not phenyl when G is O and R₁₅ is ethyl. In certain embodiments, the acid halide is not benzoyl chloride when G is O and R₁₅ is ethyl.

In certain embodiments, the aforesaid acylation reaction B proceeds in the absence of bases.

In certain embodiments, the aforesaid acylation reaction B proceeds in the presence of a suitable non-nucleophilic base, such as potassium t-butoxide, sodium hydride, lithium diisopropylamide (“LDA”), lithium hexamethyldisilazide (“LHMDS”), potassium hexamethyldidisilazide (“KHMDS”), sodium or lithium tetramethylpiperidine, or related strong bases.

Preferably the aforesaid acylation reaction B is carried out in the presence of a suitable solvent such as, for example, hydrocarbon solvents, such as benzene, toluene, xylene, or cyclohexane; halogenated hydrocarbons, such as chlorobenzene, dichloroethane, methylene chloride, chloroform, or carbon tetrachloride; carbon disulfide; dimethylformamide; ethereal solvents, like tetrahydrofuran and diethylether; or dioxane; and the like.

In certain embodiments, it is preferable to employ 1 mole to excessive mole of the aforesaid base to 1 mole of a compound of the general formula (II).

Reaction temperature is usually −60° C. to 100° C., preferably −40° C. to 80° C.

In certain embodiments of the present invention, the compound of the general formula (IV) as describe above is prepared generally with a compound of formula (I) to obtain a compound of formula (IV) through the following reaction scheme:

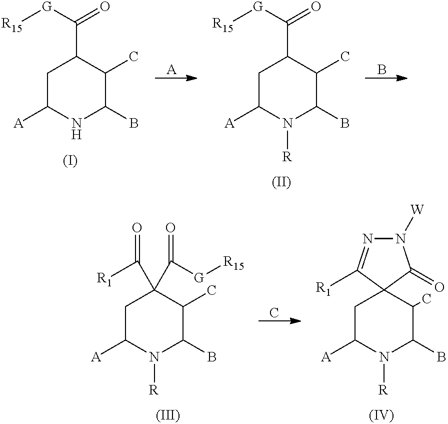

As demonstrated above, the compound of Formula (I) is preferably subject to a reaction A, forming a compound of Formula (II), which is then subject to a reaction B, forming a compound of Formula (III), which is then subject to a reaction C forming a compound of Formula (IV).

In certain embodiments, reaction A is a reductive amination reaction. In alternative embodiments, reaction A is an alkylation reaction. In yet further embodiments, reaction A is an acylation reaction.

In certain embodiments, wherein reaction A is a reductive amination reaction, a compound of formula (I)

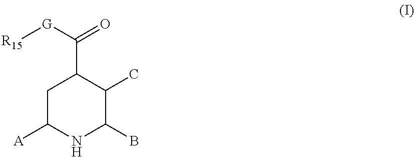

is reacted with a compound of the formula

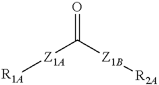

wherein Z_{1A} and Z_{1B} are the same or different and are independently selected from the group consisting of a bond, straight or branched C₁₋₆ alkylene, —NH—, —CH₂O—, —CH₂NH—, —CH₂N(CH₃)—, —NHCH₂—, —CH₂CONH—, —NHCH₂CO—, —CH₂CO—, —COCH₂—, —CH₂COCH₂—, —CH(CH₃)—, —CH═, —O— and —HC═CH—, wherein the carbon and/or nitrogen atoms are unsubstituted or substituted with one or more lower alkyl, hydroxy, halo or alkoxy group;

R_{1A}, and R_{2A} are the same or different and are independently selected from the group consisting of hydrogen, C₁₋₁₀ alkyl, C₃₋₁₂cycloalkyl, C₂₋₁₀alkenyl, amino, C₁₋₁₀alkylamino-, C₃₋₁₂cycloalkylamino-, —COOV₁, —C₁₋₄COOV₁, cyano, cyanoC₁₋₁₀alkyl-, cyanoC₃₋₁₀cycloalkyl-, NH₂SO₂—, NH₂SO₂C₁₋₄alkyl-, NH₂SOC₁₋₄alkyl-, aminocarbonyl-, C₁₋₄alkylaminocarbonyl-, diC₁₋₄alkylaminocarbonyl-, benzyl, C₃₋₁₂ cycloalkenyl-, a monocyclic, bicyclic or tricyclic aryl or heteroaryl ring, a hetero-monocyclic ring, a hetero-bicyclic ring system, and a spiro ring system of the formula (V):

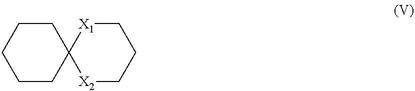

wherein X₁ and X₂ are independently selected from the group consisting of NH, O, S and CH₂; and wherein said alkyl, cycloalkyl, alkenyl, C₁₋₁₀alkylamino-, C₃₋₁₂cycloalkylamino-, or benzyl of R₁ is optionally substituted with 1-3 substituents selected from the group consisting of halogen, hydroxy, C₁₋₁₀ alkyl, C₁₋₁₀ alkoxy, nitro, trifluoromethyl-, cyano, —COOV₁, —C₁₋₄COOV₁, cyanoC₁₋₁₀alkyl-, —C₁₋₅(═O)W₁, —C₁₋₅NHS(═O)₂W₁, —C₁₋₅NHS(═O)W₁, a 5-membered heteroaromaticC₀₋₄alkyl-, phenyl, benzyl, benzyloxy, said phenyl, benzyl, and benzyloxy optionally being substituted with 1-3 substituents selected from the group consisting of halogen, C₁₋₁₀ alkyl-, C₁₋₁₀ alkoxy-, and cyano; and wherein said C₃₋₁₂ cycloalkyl, C₃₋₁₂ cycloalkenyl, monocyclic, bicyclic or tricyclic aryl, heteroaryl ring, hetero-monocyclic ring, hetero-bicyclic ring system, or spiro ring system of the formula (V) is optionally substituted with 1-3 substituents selected from the group consisting of halogen, C₁₋₁₀ alkyl, C₁₋₁₀ alkoxy, nitro, trifluoromethyl-, phenyl, benzyl, phenyloxy and benzyloxy, wherein said phenyl, benzyl, phenyloxy or benzyloxy is optionally substituted with 1-3 substituents selected from the group consisting of halogen, C₁₋₁₀ alkyl, C₁₋₁₀ alkoxy, and cyano.

In certain preferred embodiments, the R_{1A} alkyl is methyl, ethyl, propyl, butyl, pentyl, or hexyl.

In certain preferred embodiments, the R_{1A} cycloalkyl is cyclohexyl, cycloheptyl, cyclooctyl, cyclononyl, cyclodecyl, or norbornyl.

In other preferred embodiments, the R_{1A} bicyclic ring system is naphthyl. In other preferred embodiments the R_{1A} bicyclic ring system is tetrahydronaphthyl, or decahydronaphthyl and the R_{1A} tricyclic ring system is dibenzocycloheptyl. In other preferred embodiments R_{1A} is phenyl or benzyl.

In other preferred embodiments, the R_{1A} bicyclic aromatic ring is a 10-membered ring, preferably quinoline or naphthyl.

In other preferred embodiments, the R_{1A} bicyclic aromatic ring is a 9-membered ring, preferably indenyl.

In certain embodiments, Z_{1A} is a bond, methyl, or ethyl.

In certain embodiments, the Z_{1A} group is maximally substituted as not to have any hydrogen substitution on the base Z_{1A} group. For example, if the base Z_{1A} group is —CH₂—, substitution with two methyl groups would remove hydrogens from the —CH₂— base Z_{1A} group.

In certain embodiments, X₁ and X₂ are both O.

In certain embodiments, Z_{1A}R_{1A} is cyclohexylethyl-, cyclohexylmethyl-, cyclopentylmethyl-, dimethylcyclohexylmethyl-, phenylethyl-, pyrrolyltrifluoroethyl-, thienyltrifluoroethyl-, pyridylethyl-, cyclopentyl-, cyclohexyl-, methoxycyclohexyl-, tetrahydropyranyl-, propylpiperidinyl-, indolylmethyl-, pyrazoylpentyl-, thiazolylethyl-, phenyltrifluoroethyl-, hydroxyhexyl-, methoxyhexyl-, isopropoxybutyl-, hexyl-, or oxocanylpropyl-.

In certain embodiments, Z_{1A}R_{1A} is —CH₂COOV₁, tetrazolylmethyl-, cyanomethyl-, NH₂SO₂-methyl-, NH₂SOmethyl-, aminocarbonylmethyl-, C₁₋₄alkylaminocarbonylmethyl-, or diC₁₋₄alkylaminocarbonylmethyl-.

In certain embodiments, Z_{1A}R_{1A} is 3,3 diphenylpropyl optionally substituted at the 3 carbon of the propyl with —COOV₁, tetrazolylC₀₋₄alkyl-, cyano-, aminocarbonyl-, C₁₋₄alkylaminocarbonyl-, or diC₁₋₄alkylaminocarbonyl-.

In alternate embodiments; Z_{1A}R_{1A} can be

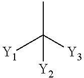

wherein

Y₁, Y₂ and Y₃ are as defined above.

In embodiments wherein reaction A is a reductive amination reaction, the reaction is preferably carried out in the presence of an acid.

Suitable acids are all inorganic and organic protonic and Lewis acids, and also all polymeric acids. These include, for example, hydrogen chloride, hydrogen bromide, sulphuric acid, formic acid, acetic acid, trifluoroacetic acid, methanesulphonic acid, trifluoromethanesulphonic acid, toluenesulphonic acid, boron trifluoride (also as etherate), boron tribromide, aluminium trichloride, zinc chloride, iron(III) chloride, antimony pentachloride, acidic ion exchangers, acidic alumina and acidic silica gel.

Preferably the process of reductive amination of reaction A is carried out in a suitable solvent such as, for example, water, an organic solvent or mixtures thereof. Examples of organic solvents include, for example, alcohols such as methanol, ethanol, n- or i-propanol, n-, sec- or tert-butanol, ethanediol, propane-1,2-diol, ethoxyethanol, methoxyethanol, diethylene glycol monomethyl ether, diethylene glycol monoethyl ether; and mixtures thereof. Particularly preferred solvents in this case are water or alcohols such as methanol, ethanol, n- or i-propanol, n-, i-, sec- or tert-butanol, ethanediol, propane-1,2-diol, ethoxyethanol, methoxyethanol, diethylene glycol monomethyl ether, diethylene glycol monoethyl ether, and mixtures thereof.

Suitable reducing agents for inclusion in the reduction amination reaction are, for example, sodium borohydride, potassium borohydride, sodium cyanoborohydride, tetramethylammonium borohydride, and the like.

In certain embodiments, it is preferable to employ 1 mole to excessive mole of the aforesaid reducing agent to 1 mole of a compound of the general formula (I).

Reaction temperature is usually −20° C. to 150° C., preferably 0° C. to 100° C.

Preferably the reaction causes the formation of a compound of Formula (II)

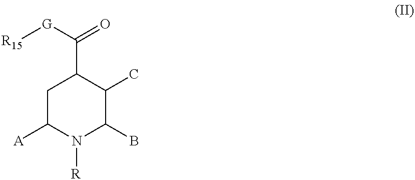

wherein R, A, B, C, G and R₁₅ are selected from the groups as disclosed above.

Alternatively, wherein reaction A is an alkylation reaction, a compound of formula (I):

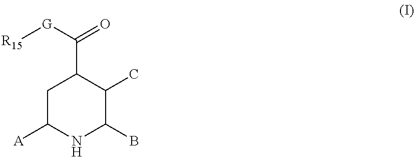

is reacted with a compound of the formula

R—X

wherein R is selected from the group as described above. X is a halogen, preferably X is Br or Cl.

In embodiments wherein reaction A is an alkylation reaction, the reaction is preferably carried out in the presence of a base. Suitable bases are all customary inorganic or organic bases. These preferably include alkaline earth metal or alkali metal hydrides, hydroxides, amides, alkoxides, acetates, carbonates or bicarbonates such as, for example, sodium hydride, sodium amide, sodium methoxide, sodium ethoxide, potassium tert-butoxide, sodium hydroxide, potassium hydroxide, ammonium hydroxide, sodium acetate, potassium acetate, calcium acetate, ammonium acetate, sodium carbonate, potassium carbonate, potassium bicarbonate, sodium bicarbonate or ammonium carbonate, and tertiary amines such as trimethylamine, triethylamine, tributylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene (DBU). Particularly preferred bases are sodium methoxide, sodium ethoxide, potassium tert-butoxide, sodium hydroxide, potassium hydroxide, ammonium hydroxide, and tertiary amines such as trimethylamine, triethylamine, tributylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene (DBU).

Preferably the process of alkylation of reaction A is carried out in a suitable solvent such as, for example, include alcohol solvents, such as, methanol, ethanol, isopropyl alcohol, or n-butanol; ketone solvents, such as methyl isobutyl ketone and methyl ethyl ketone; hydrocarbon solvents, such as benzene, toluene, or xylene; halogenated hydrocarbons, such as, chlorobenzene or methylene chloride; or dimethylformamide; and the like.

Reaction temperature can be −20° C. to 150° C., or 0° C. to 100° C.

The reaction pressure can be at standard atmosphere or under pressure, e.g., up to 45 psi.

Preferably the reaction causes the formation of a compound of Formula (II).

Alternatively, where reaction A is an acylation reaction, a compound of formula (I)

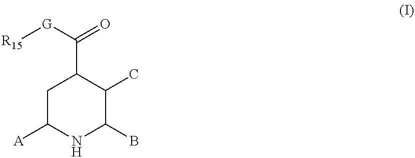

is reacted with a compound of the formula below

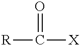

wherein R and X are selected from the groups as disclosed above.

The aforesaid reaction can proceed in the absence or presence of a base. Certain bases which may be useful in accordance with this reaction are any of those listed above.

Suitable solvents for the acylation reaction of reaction A include for example, hydrocarbon solvents, such as benzene, toluene, xylene, or cyclohexane; halogenated hydrocarbons, such as chlorobenzene, dichloroethane, methylene chloride, chloroform, or carbon tetrachloride; carbon disulfide; dimethylformamide; ethereal solvents, like tetrahydrofuran and diethylether; or dioxane; and the like.

Reaction temperature can be −60° C. to 100° C., or −40° C. to 80° C.

The reaction pressure can be at standard atmosphere or under pressure, e.g., up to 45 psi.

Preferably the reaction causes the formation of a compound of Formula (II).

After reaction A the product is preferably quenched by the addition of water and a base (e.g., NaOH) bringing the pH 10. The mixture is then extracted (e.g., with Et₂O (preferably 2 times)) and dried.

The process of the present invention further includes subjecting formula (II) to reaction B as described above, which is an acylation reaction forming a compound of the formula (III).

The compound of formula (III) is preferably then subjected to reaction C as described above, which is a reduction and cyclization reaction by reacting the compound of formula (III) with a hydrazine or substituted hydrazine (e.g., hydrazine hydrate) to form the compound of formula (IV).

The following example illustrates various aspects of the present invention, and is not to be construed to limit the claims in any manner whatsoever.

### Example 1

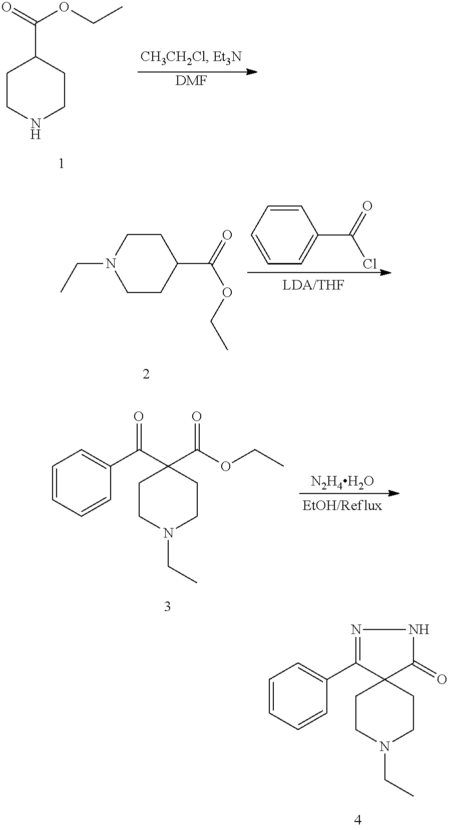

To a solution of 1 (1 eq) and triethylamine (1 eq) in dimethylformamide, is added 1 eq of ethylchloride in one portion. The mixture is stirred and heated at 80° C. over night to give a solution of 2. TLC indicates the reaction is complete.

To a solution of freshly prepared LDA in THF (1.1 eq) at −40° C. is added the solution of 2 (1 eq). The reaction mixture is allowed to warm to RT and stirred for 1 hr. After cooling to −20° C., a solution of benzoyl chloride (1.2 eq) in THF is added dropwise. After stirring at −20° C. for 1 hr and at RT for 16 hr, the reaction mixture is poured into water and extracted with ethyl acetate. The organic extracts are washed with saturated ammonium chloride, brine, dried over MgSO₄, filtered and the solvent evaporated to give crude 3 as an oil, which is used without purification in the next step.

To a solution of 3 (1 eq) in ethanol is added hydrazine hydrate (3 eq). After refluxing for 12 hr, the reaction mixture is cooled to RT and the crude product is filtered. The solid is recrystallized from ethanol to give 4 as a white solid.

Other reactions can be performed by one skilled in the art wherein compound 1 has the G and R₁₅ substituents as disclosed herein, other than the ethoxy of Example 1.

### Example 2

Preparation of starting material for embodiments wherein A, B and/or C are not hydrogen can be prepared as exemplified below:

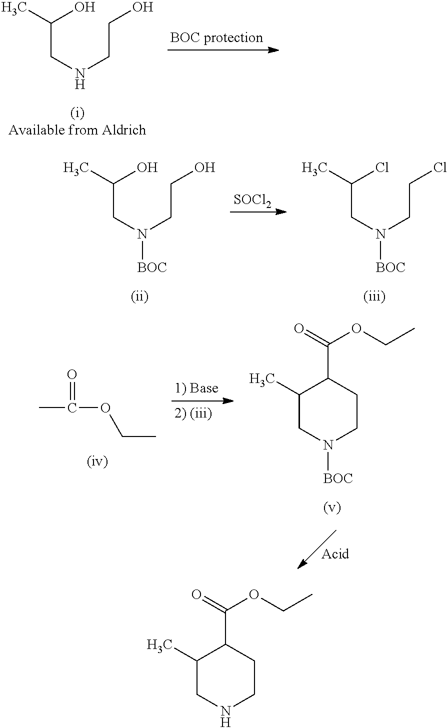

1. A process for preparing a compound of the formula (IV): 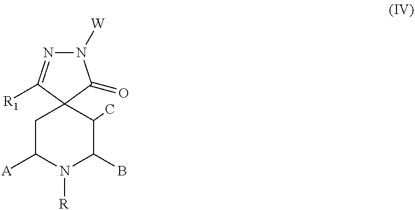
wherein
W is hydrogen, C₁₋₁₀ alkyl, C₃₋₁₂ cycloalkyl, C₃₋₁₂ cycloalkylC₁₋₄alkyl-, C₁₋₁₀ alkoxy, C₃₋₁₂ cycloalkoxy-, C₁₋₁₀ alkyl substituted with 1-3 halogen, C₃₋₁₂ cycloalkyl substituted with 1-3 halogen, C₃₋₁₂ cycloalkylC₁₋₄alkyl- substituted with 1-3 halogen, C₁₋₁₀ alkoxy substituted with 1-3 halogen, C₃₋₁₂ cycloalkoxy- substituted with 1-3 halogen, —COOV₁, —C₁₋₄COOV₁, —CH₂OH, —SO₂N(V₁)₂, hydroxyC₁₋₁₀alkyl-, hydroxyC₃₋₁₀cycloalkyl-, cyanoC₁₋₁₀alkyl-, cyanoC₃₋₁₀cycloalkyl-, —CON(V₁)₂, NH₂SO₂C₁₋₄alkyl-, NH₂SOC₁₋₄ alkyl-, sulfonylaminoC₁₋₁₀alkyl-, diaminoalkyl-, -sulfonylC₁₋₄alkyl, a 6-membered heterocyclic ring, a 6-membered heteroaromatic ring, a 6-membered heterocyclicC₁₋₄alkyl-, a 6-membered heteroaromaticC₁₋₄alkyl-, a 6-membered aromatic ring, a 6-membered aromaticC₁₋₄ alkyl-, a 5-membered heterocyclic ring optionally substituted with an oxo or thio, a 5-membered heteroaromatic ring, a 5-membered heterocyclicC₁₋₄alkyl- optionally substituted with an oxo or thio, a 5-membered heteroaromaticC₁₋₄alkyl-, —C₁₋₅(═O)W₁, —C₁₋₅(═NH)W₁, —C₁₋₅NHC(═O)W₁, —C₁₋₅NHS(═O)₂W₁, —C₁₋₅NHS(═O)W₁, wherein W₁ is hydrogen, C₁₋₁₀ alkyl, C₃₋₁₂ cycloalkyl, C₁₋₁₀ alkoxy, C₃₋₁₂ cycloalkoxy, —CH₂OH, amino, C₁₋₄alkylamino-, diC₁₋₄alkylamino-, or a 5-membered heteroaromatic ring optionally substituted with 1-3 lower alkyl;
wherein each V₁ is independently selected from H, C₁₋₆ alkyl, C₃₋₆ cycloalkyl, benzyl and phenyl;
A, B and C are independently hydrogen, C₁₋₁₀ alkyl, C₃₋₁₂ cycloalkyl, C₁₋₁₀ alkoxy, C₃₋₁₂ cycloalkoxy, —CH₂OH, —NHSO₂, hydroxyC₁₋₁₀alkyl-, aminocarbonyl-, C₁₋₄alkylaminocarbonyl-, diC₁₋₄alkylaminocarbonyl-, acylamino-, acylaminoalkyl-, amide, sulfonylaminoC₁₋₁₀alkyl-, or A-B can together form a C₂₋₆ bridge, or B-C can together form a C₃₋₇ bridge, or A-C can together form a C₁₋₅ bridge;
R is —Z—R₂; wherein Z is selected from the group consisting of a bond, straight or branched C₁₋₆ alkylene, —NH—, —CH₂O—, —CH₂NH—, —CH₂N(CH₃)—, —NHCH₂—, —CH₂CONH—, —NHCH₂CO—, —CH₂CO—, —COCH₂—, —CH₂COCH₂—, —CH(CH₃)—, —CH═, —O— and —HC═CH—, wherein the carbon and/or nitrogen atoms are unsubstituted or substituted with one or more lower alkyl, hydroxy, halo or alkoxy group;
R₂ is selected from the group consisting of hydrogen, C₁₋₁₀ alkyl, C₃₋₁₂cycloalkyl, C₂₋₁₀alkenyl, amino, C₁₋₁₀alkylamino-, C₃₋₁₂cycloalkylamino-, —COOV₁, —C₁₋₄COOV₁, cyano, cyanoC₁₋₁₀alkyl-, cyanoC₃₋₁₀cycloalkyl-, NH₂SO₂—, NH₂SO₂C₁₋₄alkyl-, NH₂SOC₁₋₄alkyl-, aminocarbonyl-, C₁₋₄alkylaminocarbonyl-, diC₁₋₄alkylaminocarbonyl-, benzyl, C₃₋₁₂ cycloalkenyl-, a monocyclic, bicyclic or tricyclic aryl or heteroaryl ring, a hetero-monocyclic ring, a hetero-bicyclic ring system, and a spiro ring system of the formula (V): 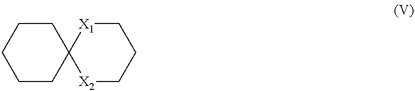
wherein X₁ and X₂ are independently selected from the group consisting of NH, O, S and CH₂; and wherein said alkyl, cycloalkyl, alkenyl, C₁₋₁₀alkylamino-, C₃₋₁₂cycloalkylamino-, or benzyl of R₁ is optionally substituted with 1-3 substituents selected from the group consisting of halogen, hydroxy, C₁₋₁₀ alkyl, C₁₋₁₀ alkoxy, nitro, trifluoromethyl-, cyano, —COOV₁, —C₁₋₄COOV₁, cyanoC₁₋₁₀alkyl-, —C₁₋₅(═O)W₁, —C₁₋₅NHS(═O)₂W₁, —C₁₋₅NHS(═O)W₁, a 5-membered heteroaromaticC₀₋₄alkyl-, phenyl, benzyl, benzyloxy, said phenyl, benzyl, and benzyloxy optionally being substituted with 1-3 substituents selected from the group consisting of halogen, C₁₋₁₀ alkyl-, C₁₋₁₀ alkoxy-, and cyano; and wherein said C₃₋₁₂ cycloalkyl, C₃₋₁₂ cycloalkenyl, monocyclic, bicyclic or tricyclic aryl, heteroaryl ring, hetero-monocyclic ring, hetero-bicyclic ring system, or spiro ring system of the formula (V) is optionally substituted with 1-3 substituents selected from the group consisting of halogen, C₁₋₁₀ alkyl, C₁₋₁₀ alkoxy, nitro, trifluoromethyl-, phenyl, benzyl, phenyloxy and benzyloxy, wherein said phenyl, benzyl, phenyloxy or benzyloxy is optionally substituted with 1-3 substituents selected from the group consisting of halogen, C₁₋₁₀ alkyl, C₁₋₁₀ alkoxy, and cyano;
R₁ is selected from the group consisting of C₁₋₈ alkyl, 5-8 membered cycloalkyl, 5-8 membered heterocyclic or a 6 membered aromatic or heteroaromatic group; and R₁ being substituted with (D)ₙ, wherein n is an integer from 0 to 3, and wherein D is selected from the group consisting of hydrogen, C₁₋₁₀ alkyl, C₃₋₁₂ cycloalkyl and halogen, said alkyl or cycloalkyl optionally substituted with an oxo, amino, alkylamino or dialkylamino group;
said process comprising:
providing a compound of the formula (III) 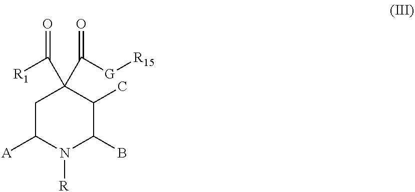
wherein A, B, C, R, and R₁ are as disclosed above, G is O or S and R₁₅ is selected from straight chained or branched C₁₋₁₀ alkyl, C₃₋₁₂ cycloalkyl, C₃₋₁₂cycloalkylC₁₋₁₀alkyl, aryl, heteroaryl, arylC₁₋₁₀alkyl or heteroarylC₁₋₁₀alkyl;
and reacting said compound of formula (III) with hydrazine, hydrates thereof, substituted hydrazine, or hydrates thereof, under conditions effective to form the compound of formula (IV): 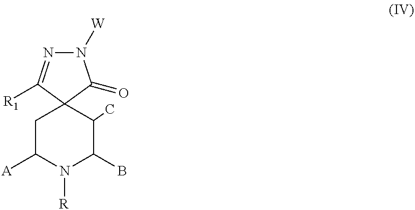
wherein A, B, C, R, R₁ and W are as disclosed above.

2. The process of claim 1, further comprising forming the compound of formula (III) by providing a compound of the formula (II): 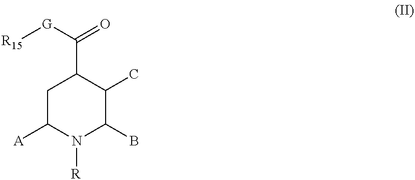
wherein A, B, C, R, G and R₁₅ are as disclosed above;
and acylating said compound of formula (II) by reacting said compound of formula (II) with a compound having the formula 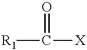
wherein R₁ is as disclosed above, and X is a halogen; under conditions effective to produce a compound of the formula (III).

3. The process of claim 2, further comprising forming the compound of formula (II) by providing a compound of formula (I): 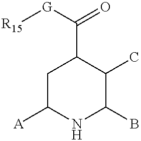
wherein A, B, C, G and R₁₅ are as disclosed above; and reacting the compound of formula (I) with a compound having the formula: 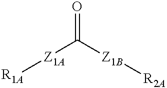
wherein Z_{1A} and Z_{1B} are the same or different and are independently selected from the group consisting of a bond, straight or branched C₁₋₆ alkylene, —NH—, —CH₂O—, —CH₂NH—, —CH₂N(CH₃)—, —NHCH₂—, —CH₂CONH—, —NHCH₂CO—, —CH₂CO—, —COCH₂—, —CH₂COCH₂—, —CH(CH₃)—, —CH═, —O— and —HC═CH—, wherein the carbon and/or nitrogen atoms are unsubstituted or substituted with one or more lower alkyl, hydroxy, halo or alkoxy group;
R_{1A} and R_{2A} are the same or different and are independently selected from the group consisting of hydrogen, C₁₋₁₀ alkyl, C₃₋₁₂cycloalkyl, C₂₋₁₀alkenyl, amino, C₁₋₁₀alkylamino-, C₃₋₁₂cycloalkylamino-, —COOV₁, —C₁₋₄COOV₁, cyano, cyanoC₁₋₁₀alkyl-, cyanoC₃₋₁₀cycloalkyl-, NH₂SO₂—, NH₂SO₂C₁₋₄alkyl-, NH₂SOC₁₋₄alkyl-, aminocarbonyl-, C₁₋₄alkylaminocarbonyl-, diC₁₋₄alkylaminocarbonyl-, benzyl, C₃₋₁₂ cycloalkenyl-, a monocyclic, bicyclic or tricyclic aryl or heteroaryl ring, a hetero-monocyclic ring, a hetero-bicyclic ring system, and a spiro ring system of the formula (V): 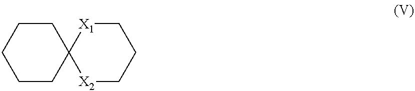
wherein X₁ and X₂ are as disclosed above;
under conditions effective to produce the compound of formula (II).

4. The process of claim 2, further comprising forming the compound of formula (II) by providing a compound of formula (I): 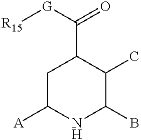
wherein A, B, C, G and R₁₅ are as disclosed above; and reacting said compound of formula (I) with a compound having the formula:
R—X
wherein R is as disclosed above and X is a halogen;
under conditions effective to produce a compound of the formula (II).

5. The process of claim 2, further comprising forming the compound of formula (II) by providing a compound of formula (I): 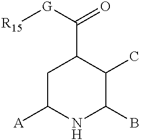
wherein A, B, C, G and R₁₅ are as disclosed above; and reacting said compound of formula (I) with a compound having the formula: 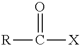
wherein R is as disclosed above and X is a halogen;
under conditions effective to produce a compound of the formula (II).

6. A process for preparing a compound of the formula (IV): 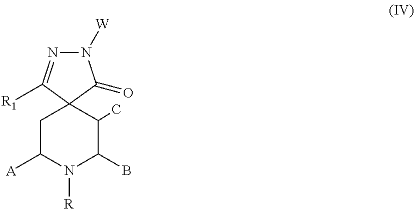
wherein
W is hydrogen, C₁₋₁₀ alkyl, C₃₋₁₂ cycloalkyl, C₃₋₁₂ cycloalkylC₁₋₄alkyl-, C₁₋₁₀ alkoxy, C₃₋₁₂ cycloalkoxy-, C₁₋₁₀ alkyl substituted with 1-3 halogen, C₃₋₁₂ cycloalkyl substituted with 1-3 halogen, C₃₋₁₂ cycloalkylC₁₋₄alkyl- substituted with 1-3 halogen, C₁₋₁₀ alkoxy substituted with 1-3 halogen, C₃₋₁₂ cycloalkoxy- substituted with 1-3 halogen, —COOV₁, —C₁₋₄ COOV₁, —CH₂OH, —SO₂N(V₁)₂ hydroxyC₁₋₁₀alkyl-, hydroxyC₃₋₁₀cycloalkyl-, cyanoC₁₋₁₀alkyl-, cyanoC₃₋₁₀-cycloalkyl-, —CON(V₁)₂, NH₂SO₂C₁₋₄alkyl-, NH₂SOC₁₋₄alkyl-, sulfonylaminoC₁₋₁₀alkyl-, diaminoalkyl-, -sulfonylC₁₋₄alkyl, a 6-membered heterocyclic ring, a 6-membered heteroaromatic ring, a 6-membered heterocyclicC₁₋₄alkyl-, a 6-membered heteroaromaticC₁₋₄alkyl-, a 6-membered aromatic ring, a 6-membered aromaticC₁₋₄ alkyl-, a 5-membered heterocyclic ring optionally substituted with an oxo or thio, a 5-membered heteroaromatic ring, a 5-membered heterocyclicC₁₋₄alkyl- optionally substituted with an oxo or thio, a 5-membered heteroaromaticC₁₋₄alkyl-, —C₁₋₅(═O)W₁, —C₁₋₅(═NH)W₁, —C₁₋₅NHC(═O)W₁, —C₁₋₅NHS(═O)₂W₁, —C₁₋₅NHS(═O)W₁, wherein W₁ is hydrogen, C₁₋₁₀ alkyl, C₃₋₁₂ cycloalkyl, C₁₋₁₀ alkoxy, C₃₋₁₂ cycloalkoxy, —CH₂OH, amino, C₁₋₄alkylamino-, diC₁₋₄alkylamino-, or a 5-membered heteroaromatic ring optionally substituted with 1-3 lower alkyl;
wherein each V₁ is independently selected from H, C₁₋₆ alkyl, C₃₋₆ cycloalkyl, benzyl and phenyl;
A, B and C are independently hydrogen, C₁₋₁₀ alkyl, C₃₋₁₂ cycloalkyl, C₁₋₁₀ alkoxy, C₃₋₁₂ cycloalkoxy, —CH₂OH, —NHSO₂, hydroxyC₁₋₁₀alkyl-, aminocarbonyl-, C₁₋₄alkylaminocarbonyl-, diC₁₋₄alkylaminocarbonyl-, acylamino-, acylaminoalkyl-, amide, sulfonylaminoC₁₋₁₀alkyl-, or A-B can together form a C₂₋₆ bridge, or B-C can together form a C₃₋₇ bridge, or A-C can together form a C₁₋₅ bridge;
R is —Z—R₂; wherein Z is selected from the group consisting of a bond, straight or branched C₁₋₆ alkylene, —NH—, —CH₂O—, —CH₂NH—, —CH₂N(CH₃)—, —NHCH₂—, —CH₂CONH—, —NHCH₂CO—, —CH₂CO—, —COCH₂—, —CH₂COCH₂—, —CH(CH₃)—, —CH═, —O— and —HC═CH—, wherein the carbon and/or nitrogen atoms are unsubstituted or substituted with one or more lower alkyl, hydroxy, halo or alkoxy group; and wherein R is not an unsubstituted benzyl when G is O and R₁₅ is ethyl;
R₂ is selected from the group consisting of hydrogen, C₁₋₁₀ alkyl, C₃₋₁₂cycloalkyl, C₂₋₁₀alkenyl, amino, C₁₋₁₀alkylamino-, C₃₋₁₂cycloalkylamino-, —COOV₁, —C₁₋₄COOV₁, cyano, cyanoC₁₋₁₀alkyl-, cyanoC₃₋₁₀cycloalkyl-, NH₂SO₂—, NH₂SO₂C₁₋₄alkyl-, NH₂SOC₁₋₄alkyl-, aminocarbonyl-, C₁₋₄alkylaminocarbonyl-, diC₁₋₄alkylaminocarbonyl-, benzyl, C₃₋₁₂ cycloalkenyl-, a monocyclic, bicyclic or tricyclic aryl or heteroaryl ring, a hetero-monocyclic ring, a hetero-bicyclic ring system, and a spiro ring system of the formula (V): 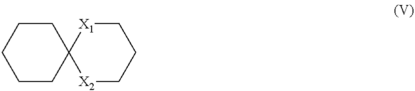
wherein X₁ and X₂ are independently selected from the group consisting of NH, O, S and CH₂; and wherein said alkyl, cycloalkyl, alkenyl, C₁₋₁₀alkylamino-, C₃₋₁₂cycloalkylamino-, or benzyl of R₁ is optionally substituted with 1-3 substituents selected from the group consisting of halogen, hydroxy, C₁₋₁₀ alkyl, C₁₋₁₀ alkoxy, nitro, trifluoromethyl-, cyano, —COOV₁, —C₁₋₄COOV₁, cyanoC₁₋₁₀alkyl-, —C₁₋₅(═O)W₁, —C₁₋₅NHS(═O)₂W₁, —C₁₋₅NHS(═O)W₁, a 5-membered heteroaromaticC₀₋₄alkyl-, phenyl, benzyl, benzyloxy, said phenyl, benzyl, and benzyloxy optionally being substituted with 1-3 substituents selected from the group consisting of halogen, C₁₋₁₀ alkyl-, C₁₋₁₀ alkoxy-, and cyano; and wherein said C₃₋₁₂ cycloalkyl, C₃₋₁₂ cycloalkenyl, monocyclic, bicyclic or tricyclic aryl, heteroaryl ring, hetero-monocyclic ring, hetero-bicyclic ring system, or spiro ring system of the formula (V) is optionally substituted with 1-3 substituents selected from the group consisting of halogen, C₁₋₁₀ alkyl, C₁₋₁₀ alkoxy, nitro, trifluoromethyl-, phenyl, benzyl, phenyloxy and benzyloxy, wherein said phenyl, benzyl, phenyloxy or benzyloxy is optionally substituted with 1-3 substituents selected from the group consisting of halogen, C₁₋₁₀ alkyl, C₁₋₁₀ alkoxy, and cyano;
R₁ is selected from the group consisting of C₁₋₈ alkyl, 5-8 membered cycloalkyl, 5-8 membered heterocyclic or a 6 membered aromatic or heteroaromatic group; and R₁ being substituted with (D)ₙ, wherein n is an integer from 0 to 3, and wherein D is selected from the group consisting of hydrogen, C₁₋₁₀ alkyl, C₃₋₁₂ cycloalkyl and halogen, said alkyl or cycloalkyl optionally substituted with an oxo, amino, alkylamino or dialkylamino group;
said process comprising:
providing a compound of the formula (III) 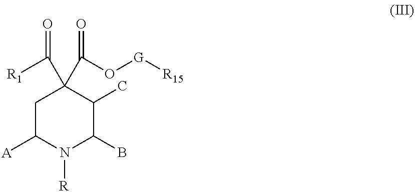
wherein A, B, C, R, and R₁ are as disclosed above, G is O or S and R₁₅ is selected from straight chained or branched C₁₋₁₀ alkyl, C₃₋₁₂ cycloalkyl, C₃₋₁₂cycloalkylC₁₋₁₀alkyl, aryl, heteroaryl, arylC₁₋₁₀alkyl or heteroarylC₁₋₁₀alkyl;
and reacting said compound of formula (III) with hydrazine, hydrates thereof, substituted hydrazine, or hydrates thereof, under conditions effective to form the compound of formula (IV): 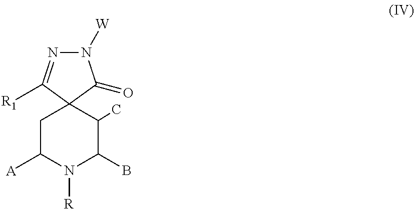
wherein A, B, C, R, R₁ and W are as disclosed above.

7. The process of claim 6, further comprising forming the compound of formula (III) by providing a compound of the formula (II): 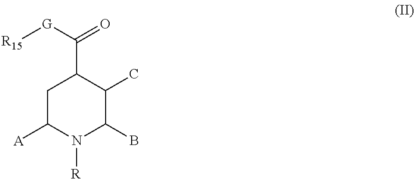
wherein A, B, C, R, G and R₁₅ are as disclosed above;
and acylating said compound of formula (II) by reacting said compound of formula (II) with a compound having the formula 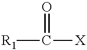
wherein R₁ is as disclosed above, and X is a halogen; under conditions effective to produce a compound of the formula (III).

8. The process of claim 7, further comprising forming the compound of formula (II) by providing a compound of formula (I): 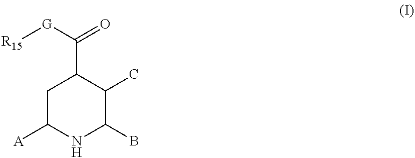
wherein A, B, C, G and R₁₅ are as disclosed above; and reacting the compound of formula (I) with a compound having the formula: 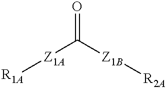
wherein Z_{1A} and Z_{1B} are the same or different and are independently selected from the group consisting of a bond, straight or branched C₁₋₆ alkylene, —NH—, —CH₂O—, —CH₂NH—, —CH₂N(CH₃)—, —NHCH₂—, —CH₂CONH—, —NHCH₂CO—, —CH₂CO—, —COCH₂—, —CH₂COCH₂—, —CH(CH₃)—, —CH═, —O— and —HC═CH—, wherein the carbon and/or nitrogen atoms are unsubstituted or substituted with one or more lower alkyl, hydroxy, halo or alkoxy group;
R_{1A} and R_{2A} are the same or different and are independently selected from the group consisting of hydrogen, C₁₋₁₀ alkyl, C₃₋₁₂cycloalkyl, C₂₋₁₀alkenyl, amino, C₁₋₁₀alkylamino-, C₃₋₁₂cycloalkylamino-, —COOV₁, —C₁₋₄COOV₁, cyano, cyanoC₁₋₁₀alkyl-, cyanoC₃₋₁₀-cycloalkyl-, NH₂SO₂—, NH₂SO₂C₁₋₄ alkyl-, NH₂SOC₁₋₄ alkyl-, aminocarbonyl-, C₁₋₄alkylaminocarbonyl-, diC₁₋₄alkylaminocarbonyl-, benzyl, C₃₋₁₂ cycloalkenyl-, a monocyclic, bicyclic or tricyclic aryl or heteroaryl ring, a hetero-monocyclic ring, a hetero-bicyclic ring system, and a spiro ring system of the formula (V): 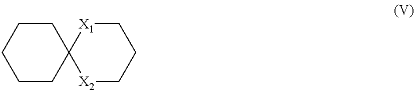
wherein X₁ and X₂ are as disclosed above; under conditions effective to produce the compound of formula (II).

9. The process of claim 7, further comprising forming the compound of formula (II) by providing a compound of formula (I): 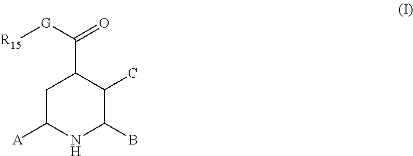
wherein A, B, C, G and R₁₅ are as disclosed above; and reacting said compound of formula (I) with a compound having the formula:
R—X
wherein R is as disclosed above and X is a halogen;
under conditions effective to produce a compound of the formula (II).

10. The process of claim 7, further comprising forming the compound of formula (II) by providing a compound of formula (I): 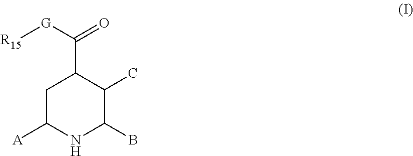
wherein A, B, C, G and R₁₅ are as disclosed above; and reacting said compound of formula (I) with a compound having the formula: 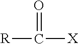
wherein R is as disclosed above and X is a halogen;
under conditions effective to produce a compound of the formula (II).

11. A process for preparing a compound of the formula (IV): 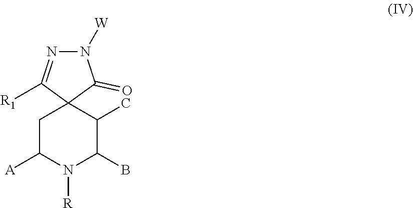
wherein
W is hydrogen, C₁₋₁₀ alkyl, C₃₋₁₂ cycloalkyl, C₃₋₁₂ cycloalkylC₁₋₄alkyl-, C₁₋₁₀ alkoxy, C₃₋₁₂ cycloalkoxy-, C₁₋₁₀ alkyl substituted with 1-3 halogen, C₃₋₁₂ cycloalkyl substituted with 1-3 halogen, C₃₋₁₂ cycloalkylC₁₋₄alkyl- substituted with 1-3 halogen, C₁₋₁₀alkoxy substituted with 1-3 halogen, C₃₋₁₂ cycloalkoxy- substituted with 1-3 halogen, —COOV₁, —C₁₋₄COOV₁, —CH₂OH, —SO₂N(V₁)₂, hydroxyC₁₋₁₀alkyl-, hydroxyC₃₋₁₀cycloalkyl-, cyanoC₁₋₁₀alkyl-, cyanoC₃₋₁₀cycloalkyl-, —CON(V₁)₂, NH₂SO₂C₁₋₄alkyl-, NH₂SOC₁₋₄alkyl-, sulfonylaminoC₁₋₁₀alkyl-, diaminoalkyl-, -sulfonylC₁₋₄alkyl, a 6-membered heterocyclic ring, a 6-membered heteroaromatic ring, a 6-membered heterocyclicC₁₋₄alkyl-, a 6-membered heteroaromaticC₁₋₄alkyl-, a 6-membered aromatic ring, a 6-membered aromaticC₁₋₄ alkyl-, a 5-membered heterocyclic ring optionally substituted with an oxo or thio, a 5-membered heteroaromatic ring, a 5-membered heterocyclicC₁₋₄alkyl- optionally substituted with an oxo or thio, a 5-membered heteroaromaticC₁₋₄alkyl-, —C₁₋₅(═O)W₁, —C₁₋₅NHC(═NH)W₁, —C₁₋₅NHC(═O)W₁, —C₁₋₅NHS(═O)₂W₁, —C₁₋₅NHS(═O)W₁, wherein W₁ is hydrogen, C₁₋₁₀ alkyl, C₃₋₁₂ cycloalkyl, C₁₋₁₀ alkoxy, C₃₋₁₂ cycloalkoxy, —CH₂OH, amino, C₁₋₄alkylamino-, diC₁₋₄alkylamino-, or a 5-membered heteroaromatic ring optionally substituted with 1-3 lower alkyl;
wherein each V₁ is independently selected from H, C₁₋₆ alkyl, C₃₋₆ cycloalkyl, benzyl and phenyl;
A, B and C are independently hydrogen, C₁₋₁₀ alkyl, C₃₋₁₂ cycloalkyl, C₁₋₁₀ alkoxy, C₃₋₁₂ cycloalkoxy, —CH₂OH, —NHSO₂, hydroxyC₁₋₁₀alkyl-, aminocarbonyl-, C₁₋₄alkylaminocarbonyl-, diC₁₋₄alkylaminocarbonyl-, acylamino-, acylaminoalkyl-, amide, sulfonylaminoC₁₋₁₀alkyl-, or A-B can together form a C₂₋₆ bridge, or B-C can together form a C₃₋₇ bridge, or A-C can together form a C₁₋₅ bridge;
R is —Z—R₂; wherein Z is selected from the group consisting of a bond, straight or branched C₁₋₆ alkylene, —NH—, —CH₂O—, —CH₂NH—, —CH₂N(CH₃)—, —NHCH₂—, —CH₂CONH—, —NHCH₂CO—, —CH₂CO—, —COCH₂—, —CH₂COCH₂—, —CH(CH₃)—, —CH═, —O— and —HC═CH—, wherein the carbon and/or nitrogen atoms are unsubstituted or substituted with one or more lower alkyl, hydroxy, halo or alkoxy group;
R₂ is selected from the group consisting of hydrogen, C₁₋₁₀ alkyl, C₃₋₁₂cycloalkyl, C₂₋₁₀alkenyl, amino, C₁₋₁₀alkylamino-, C₃₋₁₂cycloalkylamino-, —COOV₁, —C₁₋₄COOV₁, cyano, cyanoC₁₋₁₀alkyl-, cyanoC₃₋₁₀cycloalkyl-, NH₂SO₂—, NH₂SO₂C₁₋₄alkyl-, NH₂SOC₁₋₄alkyl-, aminocarbonyl-, C₁₋₄alkylaminocarbonyl-, diC₁₋₄alkylaminocarbonyl-, benzyl, C₃₋₁₂ cycloalkenyl-, a monocyclic, bicyclic or tricyclic aryl or heteroaryl ring, a hetero-monocyclic ring, a hetero-bicyclic ring system, and a spiro ring system of the formula (V): 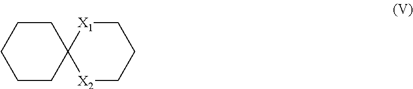
wherein X₁ and X₂ are independently selected from the group consisting of NH, O, S and CH₂; and wherein said alkyl, cycloalkyl, alkenyl, C₁₋₁₀alkylamino-, C₃₋₁₂cycloalkylamino-, or benzyl of R₁ is optionally substituted with 1-3 substituents selected from the group consisting of halogen, hydroxy, C₁₋₁₀ alkyl, C₁₋₁₀ alkoxy, nitro, trifluoromethyl-, cyano, —COOV₁, —C₁₋₄COOV₁, cyanoC₁₋₁₀alkyl, —C₁₋₅(═O)W₁, —C₁₋₅NHS(═O)₂W₁, —C₁₋₅NHS(═O)W₁, a 5-membered heteroaromaticC₀₋₄alkyl-, phenyl, benzyl, benzyloxy, said phenyl, benzyl, and benzyloxy optionally being substituted with 1-3 substituents selected from the group consisting of halogen, C₁₋₁₀ alkyl-, C₁₋₁₀ alkoxy-, and cyano; and wherein said C₃₋₁₂ cycloalkyl, C₃₋₁₂ cycloalkenyl, monocyclic, bicyclic or tricyclic aryl, heteroaryl ring, hetero-monocyclic ring, hetero-bicyclic ring system, or spiro ring system of the formula (V) is optionally substituted with 1-3 substituents selected from the group consisting of halogen, C₁₋₁₀ alkyl, C₁₋₁₀ alkoxy, nitro, trifluoromethyl-, phenyl, benzyl, phenyloxy and benzyloxy, wherein said phenyl, benzyl, phenyloxy or benzyloxy is optionally substituted with 1-3 substituents selected from the group consisting of halogen, C₁₋₁₀ alkyl, C₁₋₁₀ alkoxy, and cyano;
R₁ is selected from the group consisting of C₁₋₈ alkyl, 5-8 membered cycloalkyl, 5-8 membered heterocyclic or a 6 membered aromatic or heteroaromatic group; and R₁ being substituted with (D)ₙ, wherein n is an integer from 0 to 3, and wherein D is selected from the group consisting of hydrogen, C₁₋₁₀ alkyl, C₃₋₁₂ cycloalkyl and halogen, said alkyl or cycloalkyl optionally substituted with an oxo, amino, alkylamino or dialkylamino group;
said process comprising:
providing a compound of formula (II) 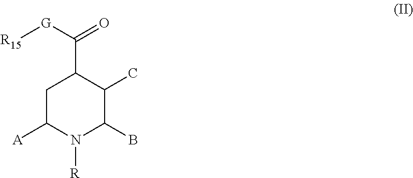
wherein A, B, C, and R are as disclosed above, G is O or S and R₁₅ is selected from straight chained or branched C₁₋₁₀ alkyl, C₃₋₁₂ cycloalkyl, C₃₋₁₂cycloalkylC₁₋₁₀alkyl, aryl, heteroaryl, arylC₁₋₁₀alkyl or heteroarylC₁₋₁₀alkyl;
and acylating said compound of formula (II) by reacting said compound of formula (II) with a compound other than benzoyl chloride when G is O and R₁₅ is ethyl having the formula 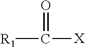
wherein R₁ is as disclosed above, and X is a halogen;
under conditions effective to produce a compound of the formula (III): 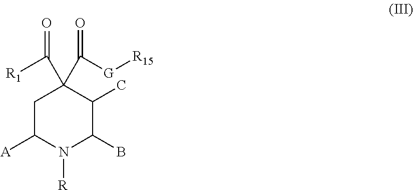
wherein A, B, C, R, R₁, G and R₁₅ are as disclosed above;
and reacting said compound of formula (III) with hydrazine, hydrates thereof, substituted hydrazine, or hydrates thereof, under conditions effective to form the compound of formula (IV): 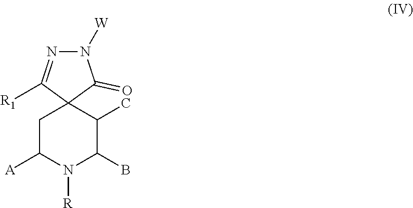
wherein A, B, C, R, R₁ and W are as disclosed above.

12. The process of claim 11, further comprising forming the compound of formula (II) by providing a compound of formula (I): 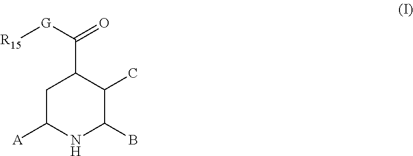
wherein A, B, C, G and R₁₅ are as disclosed above; and reacting the compound of formula (I) with a compound having the formula: 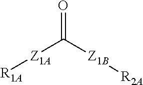
wherein Z_{1A} and Z_{1B} are the same or different and are independently selected from the group consisting of a bond, straight or branched C₁₋₆ alkylene, —NH—, —CH₂O—, —CH₂NH—, —CH₂N(CH₃)—, —NHCH₂—, —CH₂CONH—, —NHCH₂CO—, —CH₂CO—, —COCH₂—, —CH₂COCH₂—, —CH(CH₃)—, —CH═, —O— and —HC═CH—, wherein the carbon and/or nitrogen atoms are unsubstituted or substituted with one or more lower alkyl, hydroxy, halo or alkoxy group;
R_{1A} and R_{2A} are the same or different and are independently selected from the group consisting of hydrogen, C₁₋₁₀ alkyl, C₃₋₁₂cycloalkyl, C₂₋₁₀-alkenyl, amino, C₁₋₁₀alkylamino-, C₃₋₁₂cycloalkylamino-, —COOV₁, —C₁₋₄COOV₁, cyano, cyanoC₁₋₁₀alkyl-, cyanoC₃₋₁₀-cycloalkyl-, NH₂SO₂—, NH₂SO₂C₁₋₄alkyl-, NH₂SOC₁₋₄alkyl-, aminocarbonyl-, C₁₋₄alkylaminocarbonyl-, diC₁₋₄alkylaminocarbonyl-, benzyl, C₃₋₁₂ cycloalkenyl-, a monocyclic, bicyclic or tricyclic aryl or heteroaryl ring, a hetero-monocyclic ring, a hetero-bicyclic ring system, and a spiro ring system of the formula (V): 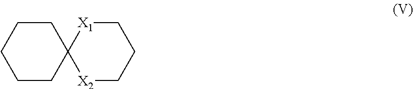
wherein X₁ and X₂ are as disclosed above;
under conditions effective to produce the compound of formula (II).

13. The process of claim 11, further comprising forming the compound of formula (II) by providing a compound of formula (I): 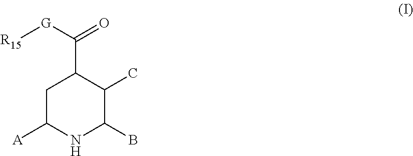
wherein A, B, C, G and R₁₅ are as disclosed above; and reacting said compound of formula (I) with a compound having the formula:
R—X
wherein R is as disclosed above and X is a halogen;
under conditions effective to produce a compound of the formula (II).

14. The process of claim 11, further comprising forming the compound of formula (II) by providing a compound of formula (I): 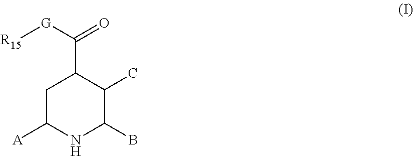
wherein A, B, C, G and R₁₅ are as disclosed above; and reacting said compound of formula (I) with a compound having the formula: 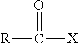
wherein R is as disclosed above and X is a halogen;
under conditions effective to produce a compound of the formula (II).

15. The process of claim 1, wherein A is hydrogen.

16. The process of claim 1, wherein B is hydrogen.

17. The process of claim 1, wherein C is hydrogen.

18. The process of claim 1, wherein A and B are hydrogen.

19. The process of claim 1, wherein A and C are hydrogen.

20. The process of claim 1, wherein B and C are hydrogen.

21. The process of claim 1, wherein A, B and C are hydrogen.

22. The process of claim 1, wherein A and B are hydrogen and C is selected from the group consisting of C₁₋₄ alkyl and hydroxyC₁₋₄alkyl.

23. The process of claim 1, wherein A and C are hydrogen and B is selected from the group consisting of C₁₋₄ alkyl and hydroxyC₁₋₄alkyl.

24. The process of claim 1, wherein B and C are hydrogen and A is selected from the group consisting of C₁₋₄ alkyl and hydroxyC₁₋₄alkyl.
